(19) United States
(12) Patent Application Publication
(10) Pub. No.: US 2023/0404533 A1
(43) Pub. Date: Dec. 21, 2023

(54) SYSTEM AND METHOD FOR AUTOMATICALLY TRACKING A MINIMAL HIATAL DIMENSION PLANE OF AN ULTRASOUND VOLUME IN REAL-TIME DURING A PELVIC FLOOR EXAMINATION

(71) Applicant: GE Precision Healthcare LLC, Wauwatosa, WI (US)

(72) Inventors: Vikram Melapudi, Bengaluru (IN); Rahul Venkataramani, Bengaluru (IN); Anuprriya Gogna, Vocklabruck (AT); Yelena Tsymbalenko, Mequon, WI (US)

(21) Appl. No.: 17/844,998

(22) Filed: Jun. 21, 2022

(51) Int. Cl. A61B 8/08; A61B 8/00

(52) U.S. Cl. CPC A61B 8/483; A61B 8/469; G06T 2207/10136; A61B 8/0883

(57) ABSTRACT

Systems and methods for automatically tracking a minimal hiatal dimension plane of an ultrasound volume in real-time during a pelvic floor examination are provided. The method includes acquiring an ultrasound volume of an anatomical region over a time period. The method includes extracting an A-plane from the ultrasound volume and displaying the A-plane. The method includes receiving an OmniView (OV) line overlaid on the A-plane. The method includes rendering an OV-plane based on a position and trajectory of the OV-line and displaying the OV-plane. The method includes automatically identifying key points in regions of interest in the A-plane. The method includes automatically tracking the key points in the regions of interest in the A-plane over the time period to automatically adjust the position and trajectory of the OV-line, the rendering the OV-plane automatically updating over the time period based on adjustments of the position and trajectory of the OV-line.

### FIELD

Certain embodiments relate to ultrasound imaging. More specifically, certain embodiments relate to a method and system for automatically tracking a minimal hiatal dimension plane of an ultrasound volume in real-time during a pelvic floor examination.

### BACKGROUND

Ultrasound imaging is a medical imaging technique for imaging organs and soft tissues in a human body. Ultrasound imaging uses real time, non-invasive high frequency sound waves to produce a series of two-dimensional (2D), three-dimensional (3D) images, and/or four-dimensional (4D) images.

Ultrasound volume acquisitions are typically described as three planes, including the A-plane, the B-plane, and the C-plane. The A-plane is the plane parallel to the acquisition plane. The B-plane is also parallel to the acquisition plane but is perpendicular to the A-plane. The C-plane, which is also known as the coronal plane, includes a thickness of two-dimensional slices parallel to and at various depths from the transducer face (i.e., perpendicular to the ultrasound beam).

Pelvic floor ultrasound examinations may be utilized to assess structural integrity of the levator ani muscle, assess possible pelvic organ prolapse, and/or assess proper functioning and strength of the pelvic floor muscles. Pelvic floor examinations are critical to evaluating the integrity of pelvic muscles and determining any preventive and/or corrective actions, including surgical intervention. During a pelvic floor examination, an ultrasound operator may perform a 4D ultrasound acquisition (i.e., 3D image acquisition over time) while the patient performs different muscle maneuvers. The ultrasound system may present the A-plane and an ultrasound operator may position an OmniView line (OV-line) or render box overlaid on the A-plane. The OV-line or render box defines the location in the 3D ultrasound volume to render as the OV-plane (i.e., corresponding to the minimal hiatal dimension view of the levator hiatus). The OV-plane may approximately correspond with the C-plane. For example, the ultrasound system may render the OV-plane from a thickness of 1-2 centimeters below the OV-line, or may render the OV-plane from image data bounded by the render box. During the pelvic floor ultrasound examination, the ultrasound system may display the A-plane and the OV-plane corresponding to the OV-line or render box in split screen.

The ultrasound operator continually monitors the dynamic images to ascertain the correctness of the maneuver and the integrity of the muscle structure and function during the pelvic floor examination. However, even though the probe is held stationary, the OV-plane corresponding with the static location of the OV-line or render box does not continue to present the correct viewing plane due to the relaxation (Valsalva phase) and contraction (contraction phase) of the muscles. Consequently, after acquisition, the operator or other medical professional performs an offline analysis to select a frame of the volume acquisition (e.g., corresponding with maximal Valsalva or maximal contraction), and reposition the OV-line or render box to obtain the rendering of the OV-plane having the minimal hiatal dimension in view such that measurements and diagnosis may be performed.

The present pelvic floor ultrasound examination workflow includes several problems and inefficiencies. For example, an ultrasound operator typically is only able to view the correct OV-plane in the selected 3D volume frame (i.e., after the 3D volume frame is selected and the OV-line or render box is repositioned). As another example, the process of selecting the 3D volume frame is subjective, which requires operator expertise and may result in fatigue, errors, inconsistencies, and/or non-robust clinical performance. In addition, the correct OV-plane image view is only available retrospectively (i.e., not in real-time during the examination), and only after adjusting of the OV-line or render box. This is because the OV-plane rendered volumes presented during the pelvic floor examination all correspond to the initially positioned OV-line or render box, despite the movement of the muscles during the examination.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for automatically tracking a minimal hiatal dimension plane of an ultrasound volume in real-time during a pelvic floor examination, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to automatically track a minimal hiatal dimension plane of an ultrasound volume in real-time during a pelvic floor examination, in accordance with various embodiments.

FIG. 2 is an illustration of an exemplary three-dimensional (3D) ultrasound volume acquisition having an A-plane, B-plane, and OV-plane, and exemplary displayed two-dimensional (2D) images including the A-plane having an OV-line and an OV-plane rendering, in accordance with various embodiments.

FIG. 3 is exemplary displays of two-dimensional (2D) A-plane images and OV-plane renderings at a first time and a second subsequent time during a pelvic floor examination, in accordance with various embodiments.

FIG. 4 is an exemplary display of a two-dimensional (2D) A-plane image having regions of interest and key points, in accordance with various embodiments.

FIG. 5 is an exemplary display of a strain image, in accordance with various embodiments.

FIG. 6 is an exemplary display of a two-dimensional (2D) A-plane image having an OV-line and an OV-plane rendering overlaid with strain information, in accordance with various embodiments.

FIG. 7 is an exemplary display of a strain graph and a strain image, in accordance with various embodiments.

FIG. 8 is an exemplary display of two-dimensional (2D) OV-plane renderings at maximal contraction phase and maximal Valsalva phase, in accordance with various embodiments.

FIG. 9 is a flow chart illustrating exemplary steps that may be utilized for automatically tracking a minimal hiatal dimension plane of an ultrasound volume in real-time during a pelvic floor examination, in accordance with various embodiments.

### DETAILED DESCRIPTION

Certain embodiments may be found in a method and system for automatically tracking a minimal hiatal dimension plane of an ultrasound volume in real-time during a pelvic floor examination. Aspects of the present disclosure have the technical effect of automatically tracking and dynamically adjusting a position and trajectory of an OmniView (OV) line in real-time during a live ultrasound volume acquisition such that a corresponding OV-image is rendered, presented, and updated throughout the live acquisition. Various embodiments have the technical effect of automatically computing strain and providing strain information with the OV-image.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, Contrast Enhanced Ultrasound (CEUS), and/or sub-modes of B-mode and/or CF such as Harmonic Imaging, Shear Wave Elasticity Imaging (SWEI), Strain Elastography, TVI, PDI, B-flow, MVI, UGAP, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphic Processing Unit (GPU), DSP, FPGA, ASIC or a combination thereof.

Although certain embodiments may describe the ultrasound examination in the context of a pelvic floor examination, for example, unless so claimed, the scope of various aspects of the present disclosure should not be limited to pelvic floor ultrasound examinations and may additionally and/or alternatively be applicable to any suitable ultrasound examination of anatomical regions having moving anatomical structures (e.g., heart, fetus, and the like).

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

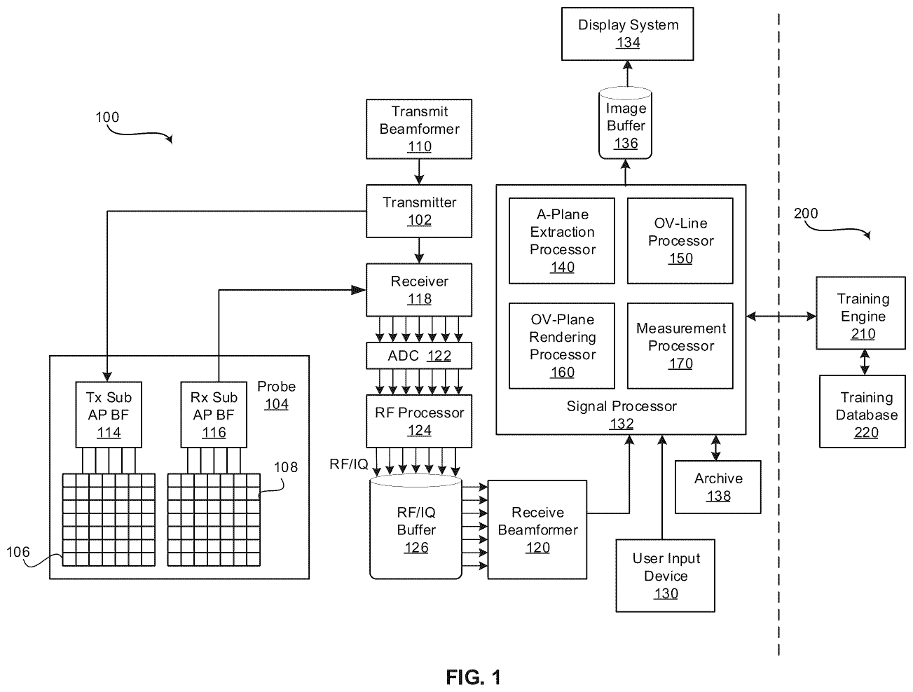

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to automatically track a minimal hiatal dimension plane of an ultrasound volume in real-time during a pelvic floor examination, in accordance with various embodiments. Referring to FIG. 1, there is shown an ultrasound system 100 and a training system 200. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two dimensional (2D) array of piezoelectric elements. Additionally and/or alternatively, the ultrasound probe 104 may be a mechanically wobbling ultrasound probe 104, which may comprise a one dimensional (1D) array of piezoelectric elements mounted on a transducer assembly movable in a single plane. For example, the transducer assembly may be movable approximately 120 to 150 degrees by a motor driving gears, belts, and/or rope to pivot an axis or hub of the transducer assembly. The ultrasound probe 104 may comprise a group of transmit transducer elements 106 and a group of receive transducer elements 108, that normally constitute the same elements. The group of transmit transducer elements 106 may emit ultrasonic signals through oil and a probe cap and into a target. In a representative embodiment, the ultrasound probe 104 may be operable to acquire ultrasound image data covering at least a substantial portion of an anatomy, such as a pelvic region or any suitable anatomical region. In an exemplary embodiment, the ultrasound probe 104 may be operated in a volume acquisition mode, where the transducer assembly of the ultrasound probe 104 is moved to acquire a plurality of parallel 2D ultrasound slices forming an ultrasound volume.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, select a position and trajectory of an OmniView (OV) line, select measurements, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134 or the ultrasound probe 104, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise an A-plane extraction processor 140, an OV-line processor 150, an OV-plane rendering processor 160, and a measurement processor 170. The signal processor 132 may be capable of receiving input information from a user input device 130 and/or archive 138, generating an output displayable by a display system 134, and manipulating the output in response to input information from a user input device 130, among other things. The signal processor 132, A-plane extraction processor 140, OV-line processor 150, OV-plane rendering processor 160, and measurement processor 170 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include an A-plane extraction processor 140 that comprises suitable logic, circuitry, interfaces and/or code that may be operable to extract and sequentially present, one at a time, the A-plane two-dimensional (2D) ultrasound image slice (i.e., the plane parallel to the acquisition plane) from a four-dimensional (4D) ultrasound volume (i.e., three-dimensional (3D) ultrasound volume over time) of a pelvic region or any suitable anatomical region. For example, the A-plane extraction processor 140 may be configured to automatically extract the A-plane from ultrasound volumes acquired over time and present the extracted A-plane at the display system 134. For example, the A-plane extraction processor 140 may be configured to generate a cine loop of extracted A-planes for playback at the display system 134. The ultrasound operator may provide pause, play, rewind, and/or fast forward instructions via the user input device to the A-plane extraction processor 140 to control playback of the cine loop. The ultrasound operator may control playback of the cine loop to view pelvic muscles at maximal Valsalva and maximal contraction for measurement. The A-plane extraction processor 140 may be configured to store the extracted A-plane images and/or cine loop at archive 138 and/or any suitable data storage medium.

The signal processor 132 may include an OV-line processor 150 that comprises suitable logic, circuitry, interfaces and/or code that may be operable to receive an initial position and trajectory of an OV-line in an initial A-plane image. The OV-line processor 150 may be configured to receive a user positioning of the OV-line overlaid on the initial A-plane image via the user input device 130. For example, in a pelvic floor examination, the OV-line may be positioned to extend through the symphysis pubis (SP) and levator ani (LA). The OV-line defines the image data of the ultrasound volume to be rendered to generate the OV-plane, which may generally correspond with the C-plane (i.e., a thickness of two-dimensional slices parallel to and at various depths from the transducer face). In a pelvic floor examination, the OV-line defines the OV-plane, which corresponds with the minimal hiatal dimension plane. In various embodiments, the rendered image data may have a thickness of 1-2 centimeters (cm) below the OV-line. The OV-line processor 150 is configured to provide the initial position and trajectory of the OV-line to the OV-plane rendering processor 160, such that the OV-plane rendering processor 160 may render and display the OV-plane image with the corresponding A-plane image based on the OV-line. Additionally or alternatively, the OV-line processor 150 may be configured to store the position and trajectory of the OV-line and/or the A-plane image having the OV-line at archive 138 and/or any suitable data storage medium.

The OV-line processor 150 may comprise suitable logic, circuitry, interfaces and/or code that may be operable to track the OV-line to automatically adjust the position and trajectory of the OV-line in subsequent A-plane images over the acquisition time period. In this regard, the OV-line processor 150 may include, for example, artificial intelligence image analysis algorithms, computer vision algorithms, one or more deep neural networks (e.g., a convolutional neural network such as u-net) and/or may utilize any suitable form of image analysis techniques or machine learning processing functionality configured to track a position and trajectory of the OV-line in A-plane images over an acquisition time period. Additionally and/or alternatively, the artificial intelligence image analysis techniques or machine learning processing functionality configured to track the position and trajectory of the OV-line in the A-plane images may be provided by a different processor or distributed across multiple processors at the ultrasound system 100 and/or a remote processor communicatively coupled to the ultrasound system 100.

As an example, the OV-line tracking functionality may be provided as a deep neural network that may be made up of, for example, an input layer, an output layer, and one or more hidden layers in between the input and output layers. Each of the layers may be made up of a plurality of processing nodes that may be referred to as neurons. For example, the OV-line tracking functionality may include an input layer having a neuron for each pixel of an A-plane image. The output layer may have a neuron corresponding to an updated position and trajectory of the OV-line. Each neuron of each layer may perform a processing function and pass the processed ultrasound image information to one of a plurality of neurons of a downstream layer for further processing. As an example, neurons of a first layer may learn to recognize edges of structure in the extracted A-plane image. The neurons of a second layer may learn to recognize shapes based on the detected edges from the first layer. The neurons of a third layer may learn positions of the recognized shapes relative to landmarks in the extracted A-plane image. The neurons of a fourth layer may learn to recognize regions of interest in the A-plane image, such as the symphysis pubis (SP) and levator ani (LA) in an A-plane image of a pelvic floor examination. The neurons of a fifth layer may learn to recognize key points in the identified regions of interest. The key points may be landmarks or other trackable features in the A-plane image. The processing performed by the deep neural network may track an updated position and trajectory of the OV-line based on the tracked key points in the identified regions of interest in the A-plane image with a high degree of probability.

In an exemplary embodiment, the OV-line processor 150 may be configured to store the OV-line position and trajectory information and/or the A-plane image having the adjusted OV-line at archive 138 and/or any suitable storage medium. The OV-line processor 150 may be configured to provide the OV-plane rendering processor 160 with the OV-line position and trajectory information, such that the OV-plane rendering processor 160 may render the OV-plane image based on the OV-line position and trajectory information and present the rendered OV-plane at the display system 134, as described below.

The signal processor 132 may include an OV-plane rendering processor 160 that comprises suitable logic, circuitry, interfaces and/or code that may be operable to render an OV-plane based on the OV-line received from the OV-line processor 150 and present the rendered OV-plane at the display system 134. For example, the OV-plane rendering processor 160 may be configured to receive the OV-line position and trajectory information from the OV-line processor 150. The OV-plane rendering processor 160 may be configured to render a 2D projection of the 3D ultrasound volume based on the OV-line position and trajectory information. For example, the OV-plane rendering processor 160 may render the 2D projection (i.e., the OV-plane image) from a thickness of 1-2 cm of the ultrasound volume below the OV-line. The rendered OV-plane image may generally correspond to the C-plane. In a pelvic floor examination, the rendered OV-plane image corresponds with the minimum hiatal distance (MHD) plane of the levator hiatus. The OV-plane rendering processor 160 continues to render and present the OV-plane image based on any adjustments to the position and trajectory of the OV-line during the acquisition time period. The OV-plane rendering processor 160 may be configured to present the rendered OV-plane image with the corresponding A-plane image having the OV-line. For example, the A-plane image having the OV-line and the OV-plane image may be presented in a split-screen view at the display system 134. The OV-plane rendering processor 160 may be configured to store the rendered OV-planes at archive 138 and/or any suitable data storage medium.

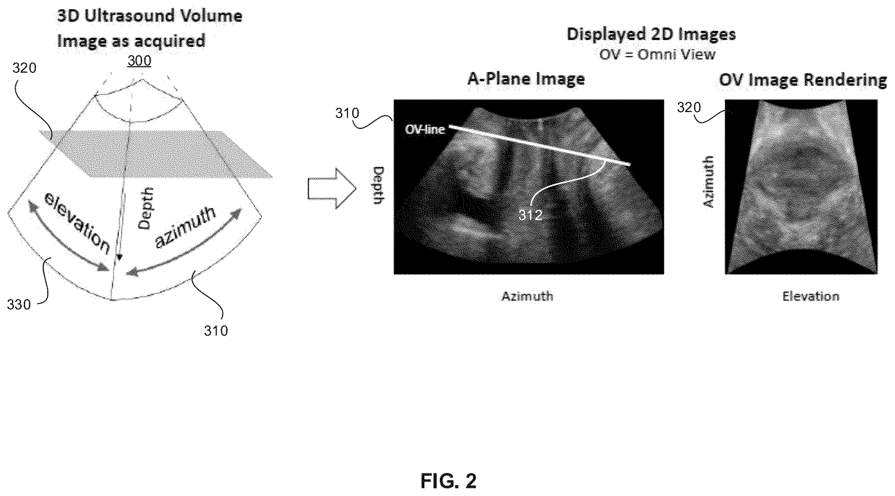

FIG. 2 is an illustration of an exemplary three-dimensional (3D) ultrasound volume acquisition 300 having an A-plane 310, B-plane 330, and OV-plane 320, and exemplary displayed two-dimensional (2D) images including the A-plane 310 having an OV-line 312 and an OV-plane rendering 320, in accordance with various embodiments. Referring to FIG. 2, the 3D ultrasound volume 300 may comprise an A-plane 310, B-plane 330, and an OV-plane 320. The OV-plane 320 may generally correspond with a C-plane (i.e., a thickness of 2D slices parallel to and at various depths from the transducer face). The OV-plane 320 is rendered and presented based on a position and trajectory of an OV-line 312. The OV-line 312 may initially be positioned in the A-plane image 310 via the user input device 130. The position and trajectory of the OV-line 312 may be automatically tracked and adjusted in subsequent A-plane images 310 extracted from ultrasound volumes 300 acquired over time (i.e., a 4D acquisition). The OV-plane image 320 is automatically updated with adjustments to the OV-line 312 over the acquisition time period.

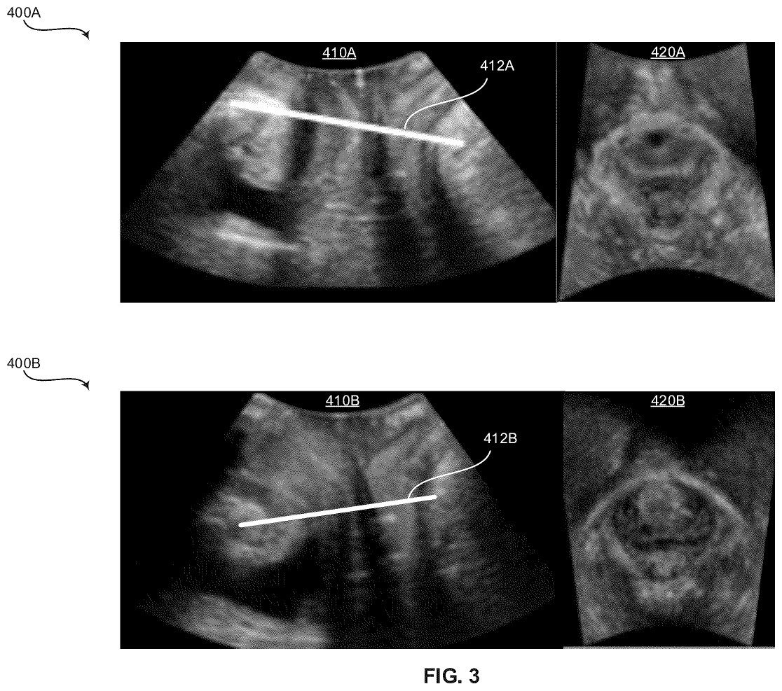

FIG. 3 is exemplary displays 400A, 400B of two-dimensional (2D) A-plane images 410A, 410B and OV-plane renderings 420A, 420B at a first time 400A and a second subsequent time 400B during a pelvic floor examination, in accordance with various embodiments. Referring to FIG. 3, an A-plane image 410A extracted from an ultrasound volume acquired at a first time 400A may be presented at a display system 134. An ultrasound operator may superimpose an OV-line 412A on the A-plane image 410A and an OV-plane rendering processor 160 may render and present an OV-plane image 420A corresponding with the OV-line 412A position and trajectory in the A-plane 410A. In various embodiments, 10-30 ultrasound volumes may be acquired over time (i.e., 4D acquisition) during the pelvic floor examination. The A-plane image 410 is extracted from each ultrasound volume and the position and trajectory of the OV-line 412 is automatically tracked and adjusted based on movement in the anatomical region (e.g., as a patient performs contraction and Valsalva muscle maneuvers during the pelvic floor examination). The OV-plane image 420B is rendered and presented based on the adjusted OV-line 412B in an A-plane image 410B extracted from an ultrasound volume acquired at the second subsequent time. Due to the dynamic tracking and adjustment of the OV-line 412A, 412B throughout the pelvic floor examination, the correct OV-plane image 420A, 420B illustrating the minimum hiatal distance (MHD) plane of the levator hiatus is rendered and presented throughout the pelvic floor examination.

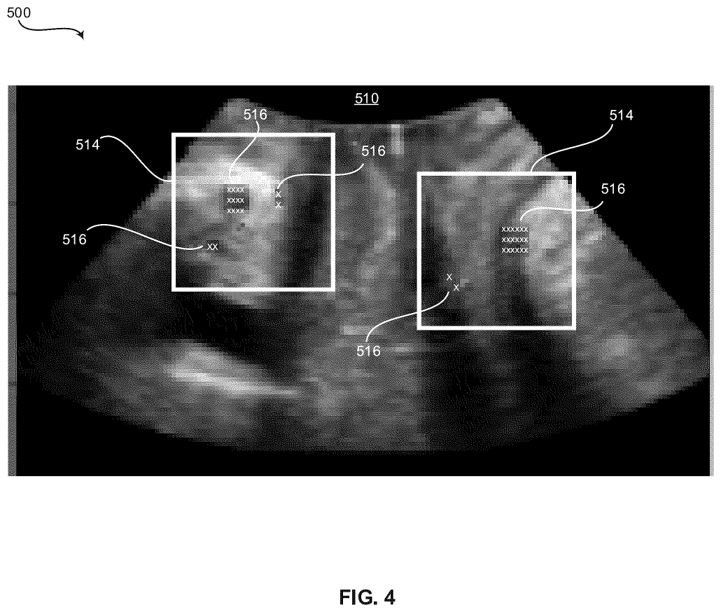

FIG. 4 is an exemplary display 500 of a two-dimensional (2D) A-plane image 510 having regions of interest 514 and key points 516, in accordance with various embodiments. Referring to FIG. 4, the OV-line processor 150 may be configured to automatically identify regions of interest 514 in the extracted A-plane images 510. For example, in a pelvic floor examination, the OV-line is positioned to extend through the symphysis pubis (SP) and levator ani (LA) regions of the pelvic region. The OV-line processor 150 may be configured to automatically identify the symphysis pubis (SP) and levator ani (LA) as the regions of interest 514 in the A-plane image 510. The OV-line processor 150 may be configured to automatically select key points 516 in the identified regions of interest 514. The key points 516 may correspond with landmarks and/or other trackable features in the A-plane image 510. The OV-line processor 150 may be configured to automatically track the regions of interest 514 and key points 516 in subsequently acquired A-plane images 510 to adjust the position and trajectory of an OV-line.

Referring again to FIG. 1, the signal processor 132 may include a measurement processor 170 that comprises suitable logic, circuitry, interfaces and/or code that may be operable to perform measurements of one or more of the OV-plane images 320, 420A, 420B rendered and presented during the examination time period. For example, the measurement processor 170 may be configured to compute strain of pelvic floor muscles in the OV-plane image 320, 420A, 420B of the levator hiatus. As an example, the Valsalva and contraction maneuvers of a pelvic floor examination cause tissue distortion. The ultrasound signals in the OV-plane 320, 420A, 420B may be tracked to compute the strain based on speckle tracking or direct strain computation (e.g., where displacements of small signal segments between two frames are first estimated, and then strains are obtained by taking the derivatives of the displacements), for example. The measurement processor 170 may be configured to generate a strain image, a graph of the strain values, and/or may overlay the strain information (e.g., color-coded) on the OV-plane image 320, 420A, 420B. In various embodiments, the measurement processor 170 may be configured to deploy a pelvic floor disorder risk artificial intelligence model to predict a risk of developing a pelvic floor disorder. For example, the model may be trained based on strain images, B-mode images, patient demographic information (e.g., age, body mass index (BMI), medical history (e.g., number of vaginal deliveries, diabetes, etc.), and the like). The measurement processor 170 may be configured to inference the model to generate a predicted risk, which may be displayed as an icon (e.g., risk meter), level (e.g., low, medium, high), and/or number (e.g., 1-5). The predicted risk may be color-coded (e.g., high risk represented by red, medium risk represented by yellow, and low risk represented by green). The predicted risk may be displayed with one or more of the A-plane image 310, 410A, 410B having the OV-line 312, 412A, 412B, the OV-plane image 320, 420A, 420B, a strain image, a strain graph, the OV-plane image 320, 420A, 420B with overlaid strain information, and/or the like.

As another example, the measurement processor 170 may be configured to compute area, length, and height based on the contours of the levator hiatus (e.g., contours of the levator hiatus at maximal contraction and maximal Valsalva). The area, length, and height measurements for each of the maximal contraction and maximal Valsalva phases, as well as ratios of the area, length, and height measurements between the maximal contraction and maximal Valsalva phases, may be presented at the display system 134.

In various embodiments, the measurement processor 170 may be configured to present measurements from past and current examinations to evaluate the treatment and disease progression over time. The measurement processor 170 may be configured to store the measurements (e.g., strain information, strain images, strain graphs, OV-plane images with superimposed strain information, area measurements, length measurements, height measurements, ratio measurements, and the like) at archive 138 and/or any suitable data storage medium.

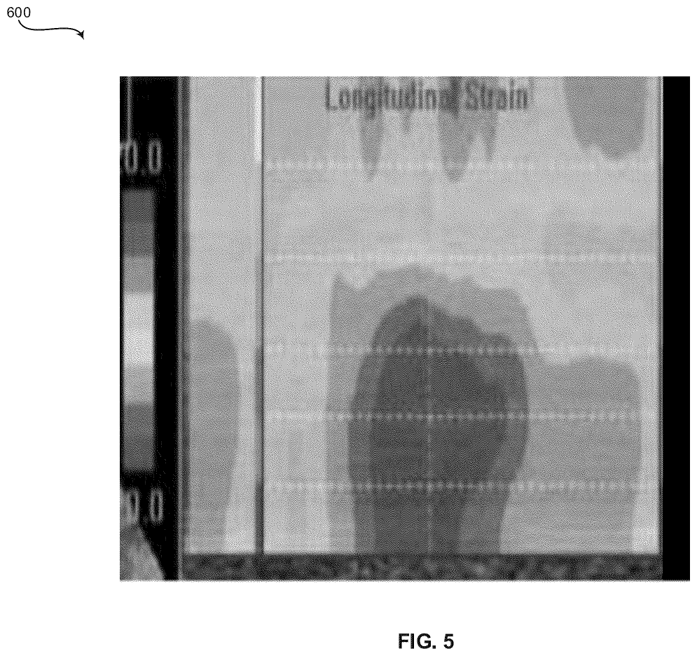

FIG. 5 is an exemplary display of a strain image 600, in accordance with various embodiments. Referring to FIG. 5, a strain image 600 of the levator hiatus is shown. The strain image 600 may be color-coded to illustrate strain values corresponding to different locations of the levator hiatus. The strain image 600 may be generated by the measurement processor 170 based on analysis of ultrasound signals (e.g., speckle tracking or direct strain computation) of the OV-plane images 320, 420A, 420B during the examination. The strain image 600 may be presented at the display system 134 by itself or with one or more of the A-plane image 310, 410A, 410B having the OV-line 312, 412A, 412B, the OV-plane image 320, 420A, 420B, a strain graph, the OV-plane image 320, 420A, 420B with overlaid strain information, area measurements, length measurements, height measurements, ratio measurements, and/or the like. The strain image 600 may be stored at archive 138 and/or any suitable data storage medium.

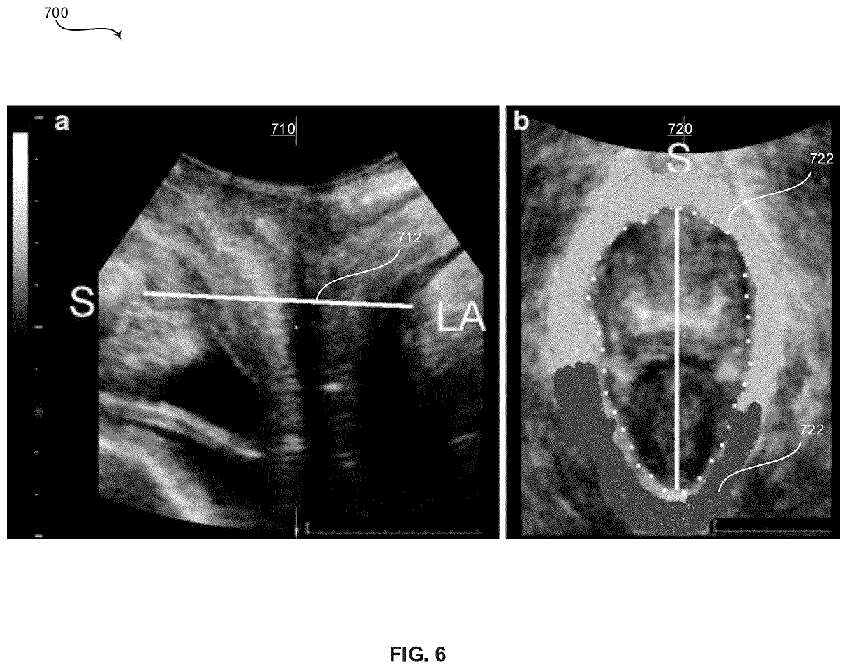

FIG. 6 is an exemplary display 700 of a two-dimensional (2D) A-plane image 710 having an OV-line 712 and an OV-plane rendering 720 overlaid with strain information 722, in accordance with various embodiments. Referring to FIG. 6, the A-plane image 710 comprises an OV-line 712 extending between the symphysis pubis (SP) and levator ani (LA) regions of the pelvic region. The OV-plane image 720 is a rendering of a thickness of 1-2 cm of image data in the acquired ultrasound volume below the OV-line 712. The measurement processor 170 computes strain information 722 by analyzing the OV-plane images 720 acquired over the examination time period based on speckle tracking or direct strain computation, for example. The computed strain information 722 is superimposed on the OV-plane image 720. In various embodiments, the strain information 722 may be color-coded based on the strain values at the different locations in the OV-plane image 720. The A-plane image 710 having the OV-line 712 and the OV-plane image 720 having the strain information 722 may be presented at the display system 134. In certain embodiments, additional measurements may be presented with the A-plane image 710 and OV-plane image 720, such as a strain graph, a strain image 600, area measurements, length measurements, height measurements, ratio measurements, and/or the like. The A-plane image 710 having the OV-line 712 and the OV-plane image 720 having the strain information 722 may be stored at archive 138 and/or any suitable data storage medium.

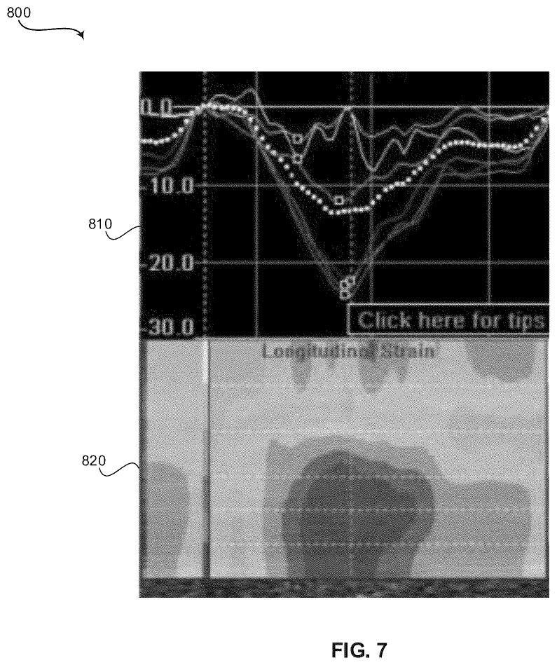

FIG. 7 is an exemplary display 800 of a strain graph 810 and a strain image 820, in accordance with various embodiments. Referring to FIG. 7, a strain graph 810 and strain image 820 may be displayed 800 at a display system 134. The strain graph 810 may illustrate strain information of various muscle locations over time. The strain image 820 may be color-coded to illustrate strain values corresponding to different muscle locations. The strain graph 810 and strain image 820 may be presented together at the display system 134. In an exemplary embodiment, additional measurements may be presented with the strain graph 810 and strain image 820, such as an A-plane image 710 having the OV-line 712, an OV-plane image 720 having the strain information 722, area measurements, length measurements, height measurements, ratio measurements, and/or the like. The strain graph 810 and strain image 820 may be stored at archive 138 and/or any suitable data storage medium.

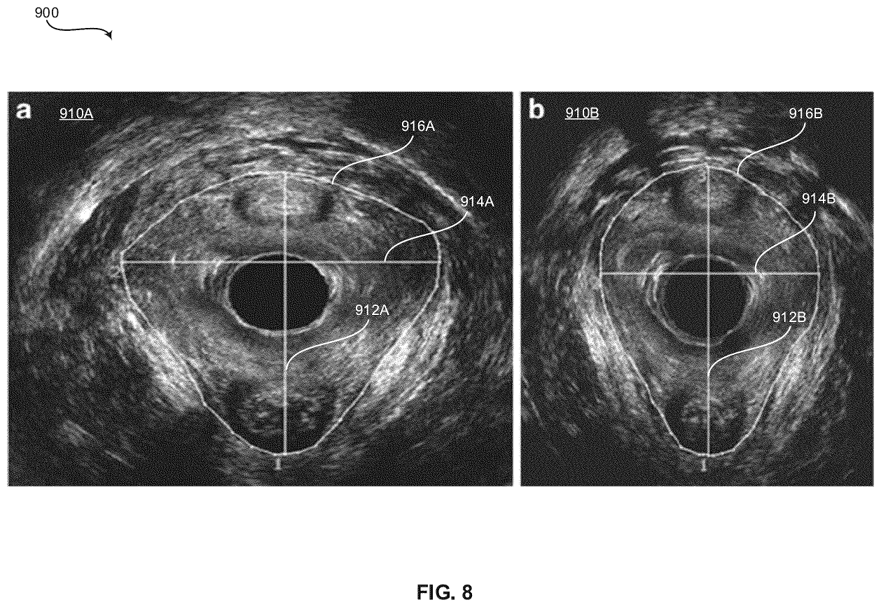

FIG. 8 is an exemplary display 900 of two-dimensional (2D) OV-plane renderings 910A, 910B at maximal contraction phase 910A and maximal Valsalva phase 910B, in accordance with various embodiments. Referring to FIG. 8, the measurement processor 170 or an ultrasound operator may select the OV-plane image having the maximal contraction phase 910A and the OV-plane image having the maximal Valsalva phase 910B. The measurement processor 170 may be configured to automatically measurement height 912A, 912B, length 914A, 914B, and area 916A, 916B of the contours of the levator hiatus at maximal contraction 910A and maximal Valsalva 910B. The measurement processor 170 may be configured to provide ratio measurements between the height 912A, 912B, length 914A, 914B, and/or area 916A, 916B of the maximal contraction 910A and maximal Valsalva 910B phases. The measurement processor 170 may be configured to present the measurements 912A, 912B, 914A, 914B, 916A, 916B and corresponding measurement values at the display system 134. In an exemplary embodiment, additional measurements may be presented with the area measurements 916A, 916B, length measurements 914A, 914B, height measurements 912A, 912B, and/or ratio measurements, such as an A-plane image 710 having the OV-line 712, an OV-plane image 720 having the strain information 722, a strain image 600, 820, a strain graph 810, and/or the like. The area measurements 916A, 916B, length measurements 914A, 914B, height measurements 912A, 912B, and/or ratio measurements may be stored at archive 138 and/or any suitable data storage medium.

Referring again to FIG. 1, the display system 134 may be any device capable of communicating visual information to a user. For example, a display system 134 may include a liquid crystal display, a light emitting diode display, and/or any suitable display or displays. The display system 134 can be operable to present the A-plane images 310, 410A, 410B, 710 having the OV-line 312, 412A, 412B, 712, the OV-plane images 320, 420A, 420B, 720, 910A, 910B, the OV-plane images 320, 420A, 420B, 720, 910A, 910B overlaid with strain information 722, strain images 600, 820, strain graphs 810, the OV-plane images 320, 420A, 420B, 720, 910A, 910B with area measurements 916A, 916B, length measurements 914A, 914B, height measurements 912A, 912B, and/or ratio measurements, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores ultrasound volumes 300, A-plane images 310, 410A, 410B, 710, OV-line 312, 412A, 412B, 712 position and trajectory information, OV-plane images 320, 420A, 420B, 720, 910A, 910B, strain information 722, strain images 600, 820, strain graphs 810, area measurements 916A, 916B, length measurements 914A, 914B, height measurements 912A, 912B, ratio measurements, instructions for extracting A-plane images 310, 410A, 410B, 710 from an ultrasound volume 300, instructions for automatically tracking and adjusting an OV-line 312, 412A, 412B, 712 in A-plane images 310, 410A, 410B, 710, instructions for rendering and displaying OV-plane images 320, 420A, 420B, 720, 910A, 910B based on the position and trajectory of the OV-line 312, 412A, 412B, 712, instructions for performing and presenting strain measurements 600, 722, 810, 820, instructions for performing and presenting area measurements 916A, 916B, length measurements 914A, 914B, height measurements 912A, 912B, and/or ratio measurements, for example.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

Still referring to FIG. 1, the training system 200 may comprise a training engine 210 and a training database 220. The training engine 210 may comprise suitable logic, circuitry, interfaces and/or code that may be operable to train the neurons of the deep neural network(s) (e.g., artificial intelligence model(s)) inferenced (i.e., deployed) by the OV-line processor 150. For example, the artificial intelligence model inferenced by the OV-line processor 150 may be trained to automatically track a position and trajectory of the OV-line 312, 412A, 412B, 712 in A-plane images 310, 410A, 410B, 710 over an acquisition time period using database(s) 220 of classified A-plane images 310, 410A, 410B, 710 of a pelvic region or any suitable anatomical region (e.g., heart, fetus, or the like).

In various embodiments, the databases 220 of training images may be a Picture Archiving and Communication System (PACS), or any suitable data storage medium. In certain embodiments, the training engine 210 and/or training image databases 220 may be remote system(s) communicatively coupled via a wired or wireless connection to the ultrasound system 100 as shown in FIG. 1. Additionally and/or alternatively, components or all of the training system 200 may be integrated with the ultrasound system 100 in various forms.

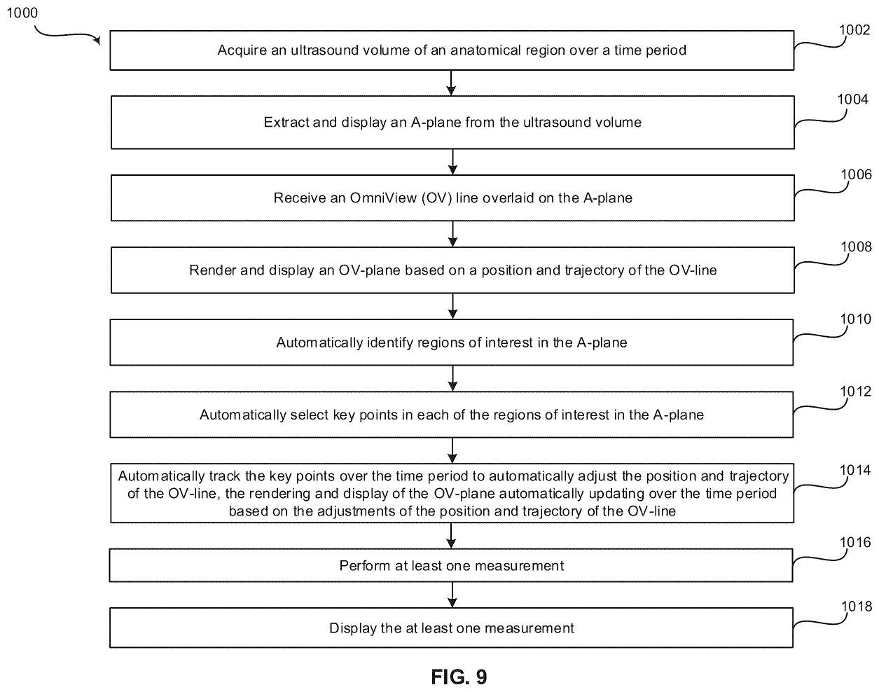

FIG. 9 is a flow chart 1000 illustrating exemplary steps 1002-1018 that may be utilized for automatically tracking a minimal hiatal dimension plane 312, 320, 412A, 420A, 412B, 420B, 712, 720, 910A, 910B of an ultrasound volume 300 in real-time during a pelvic floor examination, in accordance with various embodiments. Referring to FIG. 9, there is shown a flow chart 1000 comprising exemplary steps 1002 through 1018. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 1002, an ultrasound probe 104 of an ultrasound system 100 acquires an ultrasound volume 300 of an anatomical region over a time period. For example, the ultrasound probe 104 may be a mechanically wobbling ultrasound probe comprising a one dimensional (1D) array of piezoelectric elements mounted on a transducer assembly movable in a single plane. The ultrasound probe 104 may be operated in a volume acquisition mode, where the transducer assembly of the mechanically wobbling ultrasound probe 104 is automatically moved to acquire a plurality of parallel 2D image slices forming the ultrasound volume 300, such as an ultrasound volume 300 of a pelvic region. The ultrasound probe 104 may sequentially acquire several (e.g., 10-30) ultrasound volumes 300 over the time period (i.e., 4D acquisition). The ultrasound volumes 300 may be provided to an A-plane extraction processor 140 of a signal processor 132 and/or stored at archive 138 and/or any suitable data storage medium.

At step 1004, a signal processor 132 of the ultrasound system 100 may extract, and cause a display system 134 to present, an A-plane 310, 410A, 410B, 510, 710 from the ultrasound volume 300. For example, an A-plane extraction processor 140 of the signal processor 132 may be configured to extract and present the A-plane 310, 410A, 410B, 510, 710 from the ultrasound volume 300 at the display system 134.

At step 1006, the signal processor 132 of the ultrasound system 100 may receive an OmniView (OV) line 312, 412A, 412B, 712 overlaid on the A-plane 310, 410A, 410B, 510, 710. For example, an OV-line processor 150 of the signal processor 132 may be configured to receive a user positioning of the OV-line 312, 412A, 412B, 712 superimposed on the A-plane image 310, 410A, 410B, 510, 710 via a user input device 130. In a pelvic floor examination, for example, the OV-line 312, 412A, 412B, 712 may be positioned to extend through the symphysis pubis (SP) and levator ani (LA). The OV-line 312, 412A, 412B, 712 defines the image data of the ultrasound volume to be rendered to generate an OV-plane 320, 420A, 420B, 720, 910A, 910B. In a pelvic floor examination, the OV-line 312, 412A, 412B, 712 defines the OV-plane 320, 420A, 420B, 720, 910A, 910B, which corresponds with the minimal hiatal dimension plane. The OV-line processor 150 may be configured to provide the position and trajectory of the OV-line 312, 412A, 412B, 712 to an OV-plane rendering processor 160 of the signal processor 132 and/or store the position and trajectory of the OV-line 312, 412A, 412B, 712 and/or the A-plane image 310, 410A, 410B, 510, 710 having the OV-line 312, 412A, 412B, 712 at archive 138 and/or any suitable data storage medium.

At step 1008, the signal processor 132 of the ultrasound system 100 may render and display an OV-plane 320, 420A, 420B, 720, 910A, 910B based on a position and trajectory of the OV-line 312, 412A, 412B, 712. For example, an OV-plane rendering processor 160 of the signal processor 132 may be configured to render an OV-plane 320, 420A, 420B, 720, 910A, 910B based on the OV-line 312, 412A, 412B, 712 received from the OV-line processor 150 and present the rendered OV-plane 320, 420A, 420B, 720, 910A, 910B at the display system 134. The OV-plane rendering processor 160 may render a 2D projection (i.e., the OV-plane image 320, 420A, 420B, 720, 910A, 910B) from a thickness of 1-2 cm of the ultrasound volume 300 below the OV-line 312, 412A, 412B, 712. In a pelvic floor examination, the rendered OV-plane image 320, 420A, 420B, 720, 910A, 910B corresponds with the minimum hiatal distance (MHD) plane of the levator hiatus. The OV-plane rendering processor 160 may be configured to present the rendered OV-plane image 320, 420A, 420B, 720, 910A, 910B with the corresponding A-plane image 310, 410A, 410B, 510, 710 having the OV-line 312, 412A, 412B, 712. For example, the A-plane image 310, 410A, 410B, 510, 710 having the OV-line 312, 412A, 412B, 712 and the OV-plane image 320, 420A, 420B, 720, 910A, 910B may be presented in a split-screen view at the display system 134. The OV-plane rendering processor 160 may be configured to store the rendered OV-planes 320, 420A, 420B, 720, 910A, 910B at archive 138 and/or any suitable data storage medium.

At step 1010, the signal processor 132 of the ultrasound system 100 may automatically identify regions of interest 514 in the A-plane 310, 410A, 410B, 510, 710. For example, the OV-line processor 150 may be configured to automatically identify regions of interest 514 in the extracted A-plane images 510. For example, in a pelvic floor examination, the OV-line 312, 412A, 412B, 712 is positioned to extend through the symphysis pubis (SP) and levator ani (LA) regions of the pelvic region. The OV-line processor 150 may be configured to automatically identify the symphysis pubis (SP) and levator ani (LA) as the regions of interest 514 in the A-plane image 510.

At step 1012, the signal processor 132 of the ultrasound system 100 may automatically select key points 516 in each of the regions of interest 514 in the A-plane image 310, 410A, 410B, 510, 710. For example, the OV-line processor 150 may be configured to automatically select key points 516 in the identified regions of interest 514. The key points 516 may correspond with landmarks and/or other trackable features in the A-plane image 310, 410A, 410B, 510, 710.

At step 1014, the signal processor 132 of the ultrasound system 100 may automatically track the key points 516 over the time period to automatically adjust the position and trajectory of the OV-line 312, 412A, 412B, 712, the rendering and display of the OV-plane 320, 420A, 420B, 720, 910A, 910B automatically updating over the time period based on the adjustments of the position and trajectory of the OV-line 312, 412A, 412B, 712. For example, the OV-line processor 150 may be configured to automatically track the regions of interest 514 and key points 516 in subsequently acquired A-plane images 310, 410A, 410B, 510, 710 to adjust the position and trajectory of an OV-line 312, 412A, 412B, 712. In this regard, the OV-line processor 150 may include, for example, artificial intelligence image analysis algorithms, computer vision algorithms, one or more deep neural networks (e.g., a convolutional neural network such as u-net) and/or may utilize any suitable form of image analysis techniques or machine learning processing functionality configured to track a position and trajectory of the OV-line 312, 412A, 412B, 712 in A-plane images 310, 410A, 410B, 510, 710 over an acquisition time period. The OV-plane rendering processor 160 continues to render and present the OV-plane image 320, 420A, 420B, 720, 910A, 910B based on any automatic adjustments to the position and trajectory of the OV-line 312, 412A, 412B, 712 during the acquisition time period.

At step 1016, the signal processor 132 of the ultrasound system 100 may perform at least one measurement 600, 722, 810, 820, 912A, 912B, 914A, 914B, 916A, 916B. For example, a measurement processor 170 of the signal processor 132 may be configured to perform measurements of one or more of the OV-plane images 320, 420A, 420B, 720, 910A, 910B rendered and presented during the examination time period. As an example, the measurement processor 170 may be configured to compute strain of pelvic floor muscles in the OV-plane image 320, 420A, 420B, 720, 910A, 910B of the levator hiatus. The ultrasound signals in the OV-plane 320, 420A, 420B, 720, 910A, 910B may be tracked to compute the strain based on speckle tracking or direct strain computation (e.g., where displacements of small signal segments between two frames are first estimated, and then strains are obtained by taking the derivatives of the displacements), for example. The measurement processor 170 may be configured to generate a strain image 600, 820, a graph of the strain values 810, and/or may overlay the strain information 722 (e.g., color-coded) on the OV-plane image 320, 420A, 420B, 720, 910A, 910B. In various embodiments, the measurement processor 170 may be configured to inference a pelvic floor disorder risk artificial intelligence model to generate a predicted risk. As another example, the measurement processor 170 may be configured to compute area 916A, 916B, length 914A, 914B, height 912A, 912B, and ratios based on the contours of the levator hiatus (e.g., contours of the levator hiatus at maximal contraction and maximal Valsalva).

At step 1018, the signal processor 132 of the ultrasound system 100 may cause the display system 132 to display the at least one measurement 600, 722, 810, 820, 912A, 912B, 914A, 914B, 916A, 916B. For example, the measurement processor 170 may be configured to cause the display system 134 to present a strain image 600, 820, an OV-plane image 320, 420A, 420B, 720, 910A, 910B overlaid with strain information 722, a strain graph 810, a predicted risk, area measurements 916A, 916B, length measurements 914A, 914B, height measurements 912A, 912B, ratio measurements, and/or any suitable measurement(s).

Aspects of the present disclosure provide a method 1000 and system 100 for automatically tracking a minimal hiatal dimension plane 312, 320, 412A, 420A, 412B, 420B, 712, 720, 910A, 910B of an ultrasound volume 300 in real-time during a pelvic floor examination. In accordance with various embodiments, the method 1000 may comprise acquiring 1002, by a probe 104 of an ultrasound system 100, an ultrasound volume 300 of an anatomical region over a time period. The method 1000 may comprise extracting 1004, by at least one processor 132, 140 of the ultrasound system 100, an A-plane image 310, 410A, 410B, 510, 710 from the ultrasound volume 100. The A-plane image 310, 410A, 410B, 510, 710 is presented at a display system 134 of the ultrasound system 100. The method 1000 may comprise receiving 1006, by the at least one processor 132, 150, an OmniView (OV) line 312, 412A, 412B, 712 overlaid on the A-plane image 310, 410A, 410B, 510, 710. The method 1000 may comprise rendering 1008, by the at least one processor 132, 160, an OV-plane image 320, 420A, 420B, 720, 910A, 910B based on a position and trajectory of the OV-line 312, 412A, 412B, 712. The OV-plane image 320, 420A, 420B, 720, 910A, 910B is presented at the display system 134. The method 1000 may comprise automatically identifying 1010, 1012, by the at least one processor 132, 150, key points 516 in regions of interest 514 in the A-plane image 310, 410A, 410B, 510, 710. The method 1000 may comprise automatically tracking 1014, by the at least one processor 132, 150, the key points 516 in the regions of interest 514 in the A-plane image 310, 410A, 410B, 510, 710 over the time period to automatically adjust the position and trajectory of the OV-line 312, 412A, 412B, 712. The rendering the OV-plane image 320, 420A, 420B, 720, 910A, 910B automatically updates over the time period based on adjustments of the position and trajectory of the OV-line 312, 412A, 412B, 712.

In a representative embodiment, the anatomical region is a pelvic region. The OV-line 312, 412A, 412B, 712 overlaid on the A-plane image 310, 410A, 410B, 510, 710 may pass through a symphysis pubis and levator ani of the pelvic region. In an exemplary embodiment, the regions of interest 514 in the A-plane image 310, 410A, 410B, 510, 710 comprise the symphysis pubis and the levator ani. The OV-plane image 320, 420A, 420B, 720, 910A, 910B may correspond to a minimum hiatus distance plane 312, 320, 412A, 420A, 412B, 420B, 712, 720, 910A, 910B. In various embodiments, the automatically identifying 1010, 1012 key points 516 in the regions of interest 514 in the A-plane image 310, 410A, 410B, 510, 710 and/or the automatically tracking 1014 the key points 516 in the regions of interest 514 in the A-plane image 310, 410A, 410B, 510, 710 over the time period is performed by the at least one processor 132, 150 executing artificial intelligence. In certain embodiments, the automatically tracking 1014 the key points 516 in the regions of interest 514 in the A-plane image 310, 410A, 410B, 510, 710 over the time period is performed by the at least one processor executing computer vision. In a representative embodiment, the method 1000 comprises computing 1016, by the at least one processor 132, 170, strain based on speckle tracking or direct strain computation. The method 1000 may comprise causing, by the at least one processor 132, 170, the display system 134 to present 1018 a strain image 600, 820, the strain 722 overlaid on the OV-plane image 320, 420A, 420B, 720, 910A, 910B, and/or a strain graph 810 of the strain over time. In an exemplary embodiment, the method 1000 comprises computing 1016, by the at least one processor 132, 170, at least one measurement 600, 722, 810, 820, 912A, 912B, 914A, 914B, 916A, 916B comprising an area measurement 916A, 916B, a length measurement 914A, 914B, a height measurement 912A, 912B, and/or a ratio measurement at maximal contraction phase 910A and maximum Valsalva phase 910B. The method 1000 may comprise causing, by the at least one processor 132, 170, the display system 134 to present 1018 the measurement 600, 722, 810, 820, 912A, 912B, 914A, 914B, 916A, 916B.

Various embodiments provide a system 100 for automatically tracking a minimal hiatal dimension plane 312, 320, 412A, 420A, 412B, 420B, 712, 720, 910A, 910B of an ultrasound volume 300 in real-time during a pelvic floor examination. The ultrasound system 100 may comprise a probe 104, at least one processor 132, 140, 150, 160, 170 and a display system 134. The ultrasound probe 104 may be operable to acquire an ultrasound volume 300 of an anatomical region over a time period. The at least one processor 132, 140 may be configured to extract an A-plane image 310, 410A, 410B, 510, 710 from the ultrasound volume 300. The at least one processor 132, 150 may be configured to receive an OmniView (OV) line 312, 412A, 412B, 712 overlaid on the A-plane image 310, 410A, 410B, 510, 710. The at least one processor 132, 160 may be configured to render an OV-plane image 320, 420A, 420B, 720, 910A, 910B based on a position and trajectory of the OV-line 312, 412A, 412B, 712. The at least one processor 132, 150 may be configured to automatically identify key points 516 in regions of interest 514 in the A-plane image 310, 410A, 410B, 510, 710. The at least one processor 132, 150 may be configured to automatically track the key points 516 in the regions of interest 514 in the A-plane image 310, 410A, 410B, 510, 710 over the time period to automatically adjust the position and trajectory of the OV-line 312, 412A, 412B, 712. The at least one processor 132, 160 is configured to automatically update the OV-plane image 320, 420A, 420B, 720, 910A, 910B over the time period based on adjustments of the position and trajectory of the OV-line 312, 412A, 412B, 712. The display system 134 may be configured to present the A-plane image 310, 410A, 410B, 510, 710, the OV-line 312, 412A, 412B, 712 overlaid on the A-plane image 310, 410A, 410B, 510, 710, and the OV-plane image 320, 420A, 420B, 720, 910A, 910B.

In an exemplary embodiment, the anatomical region is a pelvic region. The OV-line 312, 412A, 412B, 712 overlaid on the A-plane image 310, 410A, 410B, 510, 710 may pass through a symphysis pubis and levator ani of the pelvic region. In various embodiments, the regions of interest in the A-plane image 310, 410A, 410B, 510, 710 comprise the symphysis pubis and the levator ani. The OV-plane image 320, 420A, 420B, 720, 910A, 910B may correspond to a minimum hiatus distance plane 312, 320, 412A, 420A, 412B, 420B, 712, 720, 910A, 910B. In certain embodiments, the at least one processor 132, 150 is configured to execute artificial intelligence to perform the automatically identifying key points 516 in the regions of interest 514 in the A-plane image 310, 410A, 410B, 510, 710 and/or the automatically tracking the key points 516 in the regions of interest 514 in the A-plane image 310, 410A, 410B, 510, 710 over the time period. In a representative embodiment, the at least one processor 132, 150 is configured to apply computer vision to perform the automatically tracking the key points 516 in the regions of interest 514 in the A-plane image 310, 410A, 410B, 510, 710 over the time period. In an exemplary embodiment, the at least one processor 132, 170 is configured to compute strain based on speckle tracking or direct strain computation. The at least one processor 132, 170 is configured to cause the display system 134 to present a strain image 600, 820, the strain 722 overlaid on the OV-plane image 320, 420A, 420B, 720, 910A, 910B, and/or a strain graph 810 of the strain over time. In various embodiments, the at least one processor 132, 170 is configured to compute at least one measurement 600, 722, 810, 820, 912A, 912B, 914A, 914B, 916A, 916B comprising an area measurement 916A, 916B, a length measurement 914A, 914B, a height measurement 912A, 912B, and/or a ratio measurement at maximal contraction phase 910A and maximum Valsalva phase 910B. The at least one processor 132, 170 is configured to cause the display system 134 to present the measurement 600, 722, 810, 820, 912A, 912B, 914A, 914B, 916A, 916B.

Certain embodiments provide a non-transitory computer readable medium having stored thereon, a computer program having at least one code section. The at least one code section is executable by a machine for causing an ultrasound system 100 to perform steps 1000. The steps 1000 may comprise receiving 1002 an ultrasound volume 300 of an anatomical region over a time period. The steps 1000 may comprise extracting 1004 an A-plane image 310, 410A, 410B, 510, 710 from the ultrasound volume 300. The A-plane image 310, 410A, 410B, 510, 710 is presented at a display system 134 of the ultrasound system 100. The steps 1000 may comprise receiving 1006 an OmniView (OV) line 312, 412A, 412B, 712 overlaid on the A-plane image 310, 410A, 410B, 510, 710. The steps 1000 may comprise rendering 1008 an OV-plane image 320, 420A, 420B, 720, 910A, 910B based on a position and trajectory of the OV-line 312, 412A, 412B, 712. The OV-plane image 320, 420A, 420B, 720, 910A, 910B is presented at the display system 134. The steps 1000 may comprise automatically identifying 1010, 1012 key points 516 in regions of interest 514 in the A-plane image 310, 410A, 410B, 510, 710. The steps 1000 may comprise automatically tracking 1014 the key points 516 in the regions of interest 514 in the A-plane image 310, 410A, 410B, 510, 710 over the time period to automatically adjust the position and trajectory of the OV-line 312, 412A, 412B, 712. The rendering 1008 the OV-plane image 320, 420A, 420B, 720, 910A, 910B automatically updates over the time period based on adjustments of the position and trajectory of the OV-line 312, 412A, 412B, 712.

In various embodiments, the anatomical region is a pelvic region. The OV-line 312, 412A, 412B, 712 overlaid on the A-plane image 310, 410A, 410B, 510, 710 passes through a symphysis pubis and levator ani of the pelvic region. The regions of interest in the A-plane image 310, 410A, 410B, 510, 710 comprise the symphysis pubis and the levator ani. The OV-plane image 320, 420A, 420B, 720, 910A, 910B corresponds to a minimum hiatus distance plane 312, 320, 412A, 420A, 412B, 420B, 712, 720, 910A, 910B. In certain embodiments, the automatically identifying 1010, 1012 key points 516 in the regions of interest 514 in the A-plane image 310, 410A, 410B, 510, 710 and/or the automatically tracking 1014 the key points 516 in the regions of interest 514 in the A-plane image 310, 410A, 410B, 510, 710 over the time period is performed by executing artificial intelligence. In a representative embodiment, the automatically tracking 1014 the key points 516 in the regions of interest 514 in the A-plane image 310, 410A, 410B, 510, 710 over the time period is performed by executing computer vision. In an exemplary embodiment, the steps 1000 may comprise computing 1016 strain based on speckle tracking or direct strain computation. The steps 1000 may comprise causing the display system 134 to present 1018 a strain image 600, 820, the strain 722 overlaid on the OV-plane image 320, 420A, 420B, 720, 910A, 910B, and/or a strain graph 810 of the strain over time. In various embodiments, the steps 1000 may comprise computing 1016 at least one measurement 600, 722, 810, 820, 912A, 912B, 914A, 914B, 916A, 916B comprising an area measurement 916A, 916B, a length measurement 914A, 914B, a height measurement 912A, 912B, and/or a ratio measurement at maximal contraction phase 910A and maximum Valsalva phase 910B. The steps 1000 may comprise causing the display system 134 to present the measurement 600, 722, 810, 820, 912A, 912B, 914A, 914B, 916A, 916B.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for automatically tracking a minimal hiatal dimension plane of an ultrasound volume in real-time during a pelvic floor examination.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
acquiring, by a probe of an ultrasound system, an ultrasound volume of an anatomical region over a time period;
extracting, by at least one processor of the ultrasound system, an A-plane image from the ultrasound volume, wherein the A-plane image is presented at a display system of the ultrasound system;
receiving, by the at least one processor, an OmniView (OV) line overlaid on the A-plane image;
rendering, by the at least one processor, an OV-plane image based on a position and trajectory of the OV-line, wherein the OV-plane image is presented at the display system;
automatically identifying, by the at least one processor, key points in regions of interest in the A-plane image; and
automatically tracking, by the at least one processor, the key points in the regions of interest in the A-plane image over the time period to automatically adjust the position and trajectory of the OV-line, the rendering the OV-plane image automatically updating over the time period based on adjustments of the position and trajectory of the OV-line.

2. The method of claim 1, wherein:
the anatomical region is a pelvic region, and
the OV-line overlaid on the A-plane image passes through a symphysis pubis and levator ani of the pelvic region.

3. The method of claim 2, wherein:
the regions of interest in the A-plane image comprise the symphysis pubis and the levator ani, and
the OV-plane image corresponds to a minimum hiatus distance plane.

4. The method of claim 1, wherein the automatically identifying key points in the regions of interest in the A-plane image and/or the automatically tracking the key points in the regions of interest in the A-plane image over the time period is performed by the at least one processor executing artificial intelligence.

5. The method of claim 1, wherein the automatically tracking the key points in the regions of interest in the A-plane image over the time period is performed by the at least one processor executing computer vision.

6. The method of claim 1, comprising:
computing, by the at least one processor, strain based on speckle tracking or direct strain computation; and
causing, by the at least one processor, the display system to present:
a strain image,
the strain overlaid on the OV-plane image, and/or
a strain graph of the strain over time.

7. The method of claim 1, comprising:
computing, by the at least one processor, at least one measurement comprising an area measurement, a length measurement, a height measurement, and/or a ratio measurement at maximal contraction phase and maximum Valsalva phase; and
causing, by the at least one processor, the display system to present the measurement.

8. A ultrasound system comprising:
an ultrasound probe operable to acquire an ultrasound volume of an anatomical region over a time period;
at least one processor configured to:
extract an A-plane image from the ultrasound volume;
receive an OmniView (OV) line overlaid on the A-plane image;
render an OV-plane image based on a position and trajectory of the OV-line;
automatically identify key points in regions of interest in the A-plane image; and
automatically track the key points in the regions of interest in the A-plane image over the time period to automatically adjust the position and trajectory of the OV-line, wherein the at least one processor is configured to automatically update the OV-plane image over the time period based on adjustments of the position and trajectory of the OV-line; and
a display system configured to present:
the A-plane image;
the OV-line overlaid on the A-plane image; and
the OV-plane image.

9. The ultrasound system of claim 8, wherein:
the anatomical region is a pelvic region, and
the OV-line overlaid on the A-plane image passes through a symphysis pubis and levator ani of the pelvic region.

10. The ultrasound system of claim 9, wherein:
the regions of interest in the A-plane image comprise the symphysis pubis and the levator ani, and
the OV-plane image corresponds to a minimum hiatus distance plane.

11. The ultrasound system of claim 8, wherein the at least one processor is configured to execute artificial intelligence to perform the automatically identifying key points in the regions of interest in the A-plane image and/or the automatically tracking the key points in the regions of interest in the A-plane image over the time period.

12. The ultrasound system of claim 8, wherein the at least one processor is configured to apply computer vision to perform the automatically tracking the key points in the regions of interest in the A-plane image over the time period.

13. The ultrasound system of claim 8, wherein the at least one processor is configured to:
compute strain based on speckle tracking or direct strain computation; and
cause the display system to present:
a strain image,
the strain overlaid on the OV-plane image, and/or
a strain graph of the strain over time.

14. The ultrasound system of claim 8, wherein the at least one processor is configured to:
compute at least one measurement comprising an area measurement, a length measurement, a height measurement, and/or a ratio measurement at maximal contraction phase and maximum Valsalva phase; and
cause the display system to present the measurement.

15. A non-transitory computer readable medium having stored thereon, a computer program having at least one code section, the at least one code section being executable by a machine for causing an ultrasound system to perform steps comprising:
receiving an ultrasound volume of an anatomical region over a time period;
extracting an A-plane image from the ultrasound volume, wherein the A-plane image is presented at a display system of the ultrasound system;
receiving an OmniView (OV) line overlaid on the A-plane image;
rendering an OV-plane image based on a position and trajectory of the OV-line, wherein the OV-plane image is presented at the display system;
automatically identifying key points in regions of interest in the A-plane image; and
automatically tracking the key points in the regions of interest in the A-plane image over the time period to automatically adjust the position and trajectory of the OV-line, the rendering the OV-plane image automatically updating over the time period based on adjustments of the position and trajectory of the OV-line.

16. The non-transitory computer readable medium of claim 15, wherein:
the anatomical region is a pelvic region,
the OV-line overlaid on the A-plane image passes through a symphysis pubis and levator ani of the pelvic region,
the regions of interest in the A-plane image comprise the symphysis pubis and the levator ani, and
the OV-plane image corresponds to a minimum hiatus distance plane.

17. The non-transitory computer readable medium of claim 15, wherein the automatically identifying key points in the regions of interest in the A-plane image and/or the automatically tracking the key points in the regions of interest in the A-plane image over the time period is performed by executing artificial intelligence.

18. The non-transitory computer readable medium of claim 15, wherein the automatically tracking the key points in the regions of interest in the A-plane image over the time period is performed by executing computer vision.

19. The non-transitory computer readable medium of claim 15, comprising:
computing strain based on speckle tracking or direct strain computation; and
causing the display system to present:
a strain image,
the strain overlaid on the OV-plane image, and/or
a strain graph of the strain over time.

20. The non-transitory computer readable medium of claim 15, comprising:
computing at least one measurement comprising an area measurement, a length measurement, a height measurement, and/or a ratio measurement at maximal contraction phase and maximum Valsalva phase; and
causing the display system to present the measurement.
